(19) United States
(12) Patent Application Publication
(10) Pub. No.: US 2017/0342460 A1
(43) Pub. Date: Nov. 30, 2017

(54) METHOD FOR SCREENING FOR BIOACTIVE NATURAL PRODUCTS

(71) Applicant: Bactevo Ltd., Cambridgeshire (GB)

(72) Inventors: David Hugh Williams, Melbourn (GB); John Richard Wain, Melbourn (GB); Stuart Robert Wood, Longstanton (GB)

(73) Assignee: Bactevo Ltd., Cambrigeshire (GB)

(21) Appl. No.: 15/617,898

(22) Filed: Jun. 8, 2017

Related U.S. Application Data
(63) Continuation of application No. PCT/GB2015/053774, filed on Dec. 9, 2015.

(30) Foreign Application Priority Data
Dec. 9, 2014 (GB) 1421850.7

(51) Int. Cl. C12Q 1/18; C12M 1/12; C12N 1/20; C12N 15/10

(52) U.S. Cl. CPC C12Q 1/18; C12N 15/1058; C12N 15/1082; C12M 25/01; C12N 15/102; C12N 1/20; C12Q 2600/136

(57) ABSTRACT

Describe is a method for screening mutant prokaryotic cells to identify producers of a cytotoxic agent active against a target cell, the method comprising the steps of: (a) providing cells of a producer prokaryotic species; (b) generating a pool of mutant producer cells by transposon mutagenesis of the cells of step (a) with an activating transposon (TnA), wherein the TnA comprises an outward-facing promoter (TnAP) capable of increasing transcription of a gene at or near its insertion site in the DNA of said producer cells; (c) co-encapsulating individual members of the pool of step (b) with one or more target cells in microdroplets, the microdroplets comprising a volume of aqueous growth media suspended in an immiscible carrier liquid, thereby generating a library of microdroplets each comprising a single mutant producer cell and one or more target cell(s); (d) incubating the microdroplet library of step (c) under conditions suitable for co-culture of the single mutant producer cell and target cell(s) to produce a library of microcultures, whereby mutant producer cells producing a cytotoxic agent active against the target cell(s) outgrow target cells in each microculture; and (e) screening the library of microcultures of step (d) for microcultures in which target cells have been outgrown or overgrown to extinction by mutant producer cells.

### FIELD OF THE INVENTION

The present invention relates to methods for screening mutant prokaryotic cells to identify producers of cytotoxic agents (such as antibiotics and anticancer agents) active against a target cell (such as pathogenic bacteria and tumour cells), and to methods of identifying a cytotoxic agent comprising such screening methods. The invention also relates to processes for producing a cytotoxic agent comprising the methods of the invention.

### BACKGROUND TO THE INVENTION

Bacteria are a major source of bioactive natural products, including antibiotics, anticancer agents, crop protection agents and immunosuppressants. For example, actinobacteria, especially Streptomyces spp., are producers of many bioactive secondary metabolites that are useful in medicine (e.g. as antibacterials, antifungals, antivirals, antithrombotics, immunomodulatory agents, anticancer agents and enzyme inhibitors) and in agriculture (e.g. as insecticides, herbicides, fungicides and growth promoting substances for plants and animals). Actinobacteria-derived antibiotics that are important in medicine include aminoglycosides, anthracyclines, chloramphenicol, macrolide and tetracyclines, while natural bacterial products such as bleomycin, doxorubicin, rapamycin and mithramycin are the basis of important anticancer therapeutics.

However, there is an urgent need for new cytotoxic agents, especially antibiotics, to counter the emergence of new pathogens and resistance to existing antimicrobial drugs, whilst the range of anticancer agents must be expanded.

Traditional screening for producers of cytotoxic agents include testing pure strains of a candidate producer for activity against target cells in solid or liquid media. In the former case, individual producing bacteria with genetic mutants can be plated onto lawns of target cells so that those producing the desired cytotoxic agents can be identified by the appearance of zones of inhibition/clearing surrounding the emergent mutant colonies. However, this technique is laborious, cannot typically be applied in the case of mammalian target cells, and is not suited to high throughput screens.

Screening mutants in liquid media is complicated by the facts that mutants of interest producing cytotoxic compounds may exhibit widely different growth rates, greatly reducing the diversity of the recovered producer mutants. Moreover, target mutants producing cytotoxic compounds may also be overgrown by “cheaters”, these being mutants which are resistant to the cytotoxic compounds produced by the target mutant producer cells but which do not themselves produce the cytotoxic agent (so enjoying a metabolic advantage reflected in a higher growth rate).

A further problem associated with screening for producer mutants in liquid culture arises from the fact that the cytotoxic compounds of interest may be produced at relatively low concentrations, and so effectively diluted out by the bulk liquid culture medium. Thus, valuable signals arising from mutant producer cells may go undetected (or be obscured by the effects of mutant producer cells secreting more potent cytotoxic agents).

A major challenge to the development of new bioactive natural products is therefore the need to screen large numbers of producer bacteria to identify those elaborating products having the desired activity, coupled with the need to secure a source of rich biological diversity at the level of the candidate producer organisms to be screened. There is also a need for methods which can be used in high-throughput, massively parallel screens so that very large numbers of different producer cell mutants can be screened against a wide range of different target cells.

### SUMMARY OF THE INVENTION

According to an aspect of the present invention, there is provided a method for screening mutant prokaryotic cells to identify producers of a cytotoxic agent active against a target cell, the method comprising the steps of:
(a) providing cells of a producer prokaryotic species;
(b) generating a pool of mutant producer cells by transposon mutagenesis of the cells of step (a) with an activating transposon (Tn_{A}), wherein the Tn_{A} comprises an outward-facing promoter (Tn_{A}P) capable of increasing transcription of a gene at or near its insertion site in the DNA of said producer cells;
(c) co-encapsulating individual members of the pool of step (b) with one or more target cells in microdroplets, the microdroplets comprising a volume of aqueous growth media suspended in an immiscible carrier liquid, thereby generating a library of microdroplets each comprising a single mutant producer cell and one or more target cell(s);
(d) incubating the microdroplet library of step (c) under conditions suitable for co-culture of the single mutant producer cell and target cell(s) to produce a library of microcultures, whereby mutant producer cells producing a cytotoxic agent active against the target cell(s) outgrow target cells in each microculture; and
(e) screening the library of microcultures of step (d) for microcultures in which target cells have been outgrown or overgrown to extinction by mutant producer cells.

The encapsulation step is preferably conducted such that each microdroplet contains a single member of the mutant pool, together with two or more (for example, about 10) target cells. Depending on the encapsulation process employed, the microdroplet library may be heterogeneous with respect to cellular content, and some microdroplets may be empty. However, all that is required is that encapsulation result in the recovery of at least some microdroplets containing a single mutant producer cell from the mutant pool along with one or more target cells.

The method of the invention permits enrichment for microcultures in which target cells have been killed and/or outgrown by mutant producer cells, which mutant cells may be subsequently isolated and analysed to identify the basis for their cytotoxic activity. Since the assay is performed in relatively small volumes, the effect of each cytotoxic compound produced by a mutant producer cell may be detected without the diluting effect of a large volume of media and/or the confounding effect of other (possibly more potent) cytotoxic agents produced by other mutant producer cells. Thus, the method is much more sensitive to signals generated by mutant producer cells.

The method also avoids the “swamping” effect of “cheaters”, these being mutant producer cells which are resistant to the cytotoxic compounds produced by the target mutant producer cells but which do not themselves produce the cytotoxic agent (so enjoying a metabolic advantage reflected in a higher growth rate). Such “cheaters” would overgrow and effectively extinguish the signal generated by mutant producers of cytotoxic agents if not effectively partitioned from individual producer cell mutants by the encapsulation step of the invention.

The method may further comprise sequencing the DNA of mutant producer cells in microdroplets in which target cells have been outgrown or overgrown to extinction by mutant producer cells during the incubation step. Such microdroplets can be isolated by various sorting techniques (see infra), and the cells can be released from the microdroplets by any convenient method (for example by the addition of surfactants, detergents, by sonication, by osmotic shock or by mechanical or physicochemical means).

DNA adjacent or near the insertion site of the Tn_{A} is preferably sequenced, for example by methods comprising the selective amplification of transposon-cellular DNA junctions.

In preferred embodiments, the sequencing comprises high-throughput massively parallel sequencing. Any such type of sequencing may be employed, for example selected from: (a) sequencing-by-synthesis (SBS) biochemistry; and/or (b) nanopore sequencing; and/or (c) tunnelling current sequencing; and/or (d) pyrosequencing; and/or (e) sequencing-by-ligation (SOLiD sequencing); and/or (f) ion semiconductor; and/or (g) mass spectrometry sequencing.

Preferably, about 25, 50, 75, 100 or greater than 100 base pairs of DNA adjacent or near the Tn_{A} insertion site are sequenced. The sequenced DNA may be 5′ and/or 3′ to the Tn_{A} insertion site.

The methods of the invention may further comprise the step of sequencing mRNA transcripts produced by Tn_{A}P in mutant producer cells in microdroplets in which target cells have been outgrown or overgrown to extinction by mutant producer cells to produce an mRNA transcript profile. In such embodiments, the mRNA transcript profile comprises a determination of:
(a) the sequences of said mRNA transcripts produced by Tn_{A}P; and/or
(b) the start and finish of mRNA transcripts produced by TnAP; and/or
(c) the lengths of said mRNA transcripts produced by Tn_{A}P; and/or
(d) the relative abundance of said mRNA transcripts produced by Tn_{A}P; and/or
(e) the site of transcription on the cellular DNA; and/or
(f) whether the mRNA transcripts produced by Tn_{A}P is sense or antisense with respect to the cellular DNA; and/or
(g) whether the mRNA transcripts produced by Tn_{A}P correspond to ORFs with respect to the cellular DNA; and/or
(h) whether the mRNA transcripts produced by Tn_{A}P encode prokaryotic proteins and/or protein domains.

The size of the microdroplets will be selected by reference to the nature of the cells (both producer and target) to be encapsulated, and the number of doublings to be achieved during the incubation step. Typically, the microdroplets are sized to provide a volume of growth medium sufficient to support 1000 cells. Thus, the microdroplets may be substantially spherical with a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm.

While the microdroplets may comprise a volume of aqueous growth media in the gel state, in preferred embodiments they comprise a volume of aqueous growth media in the liquid state. In such embodiments, the microdroplets may comprise an inner core of aqueous growth media enveloped in an outer oil shell, the carrier liquid being a continuous aqueous phase. Here, the inner aqueous core has a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm, while the outer oil shell may have a thickness of: (a) 10 μm to 200 μm; (b) 10 μm to 200 μm; (c) 10 μm to 150 μm; (d) 10 μm to 100 μm; (e) 10 μm to 50 μm; or (f) about 100 μm.

In single W/O type emulsions, the carrier liquid may be any water-immiscible liquid, for example an oil, optionally selected from: (a) a hydrocarbon oil; (b) a fluorocarbon oil; (c) an ester oil; (d) an oil having low solubility for biological components of the aqueous phase; (e) an oil which inhibits molecular diffusion between microdroplets; (f) an oil which is hydrophobic and lipophobic; (g) an oil having good solubility for gases; and/or (h) combinations of any two or more of the foregoing.

Thus, the microdroplets may be comprised in a W/O emulsion wherein the microdroplets constitute an aqueous, dispersed, phase and the carrier liquid constitutes a continuous oil phase.

In other embodiments, the microdroplets are comprised in a W/O/W double emulsion and the carrier liquid may an aqueous liquid. In such embodiments, the aqueous liquid may be phosphate buffered saline (PBS).

The microdroplets may therefore be comprised in a W/O/W double emulsion wherein the microdroplets comprise: (a) an inner core of aqueous growth media enveloped in an outer oil shell as the dispersed phase, and (b) the carrier liquid as the continuous aqueous phase.

In embodiments where the microdroplets are comprised in an emulsion, the carrier liquid may constitute the continuous phase and the microdroplets the dispersed phase, and in such embodiments the emulsion may further comprise a surfactant and optionally a co-surfactant.

The surfactant and/or co-surfactant may be located at the interface of the dispersed and continuous phases, and when the microdroplets are comprised in a W/O/W double emulsion the surfactant and/or co-surfactant may be located at the interface of aqueous core and oil shell and at the interface of the oil shell and outer continuous phase

The microdroplets may be monodispersed (as defined herein).

The co-encapsulation step may comprise mixing: (i) the pool of mutant producer cells; (ii) a population of the target cells; (iii) an aqueous growth medium; (iv) a water-immiscible liquid, for example an oil as defined herein; and (v) a surfactant, for example as defined herein, under conditions whereby a W/O type single emulsion comprising microdroplets of the aqueous growth medium dispersed in the water-immiscible liquid is formed.

In some embodiments, the W/O type single emulsion as described above is used for the incubation step. This may be preferred in circumstances where the continuous oil phase provides improved compartmentalization of the microcultures (for example, by preventing or limiting inter-culture interactions mediated by water soluble bioactive produces released during culture). In such embodiments, subsequent manipulation, screening (and in particular sorting) may be facilitated by a further, post-incubation, emulsification step wherein an aqueous carrier liquid, for example as defined herein, is mixed with the single emulsion used for the incubation step under conditions whereby a W/O/W double emulsion comprising microdroplets of the aqueous growth medium enveloped in the water-immiscible liquid and dispersed in the aqueous carrier liquid is formed.

The co-encapsulation step (c) may comprises mixing: (a) the pool of mutant producer cells; (b) a population of the target cells; (c) an aqueous growth medium; (d) a water-immiscible liquid, for example an oil as defined herein; (e) a surfactant, for example as defined herein, and (f) an aqueous carrier liquid, for example as defined herein, under conditions whereby a W/O/W double emulsion comprising microdroplets of the aqueous growth medium enveloped in the water-immiscible liquid and dispersed in the aqueous carrier liquid is formed.

Any means may be employed for the mixing step: for example, this step may comprise: (a) vortexing and/or (b) sonication; (c) homogenization; (d) pico-injection and/or (e) flow focusing.

As explained above, depending on the encapsulation process employed, the microdroplet library may be heterogeneous with respect to cellular content, and some microdroplets may be empty. In such circumstances the co-encapsulation step (c) may further comprise eliminating empty microdroplets which do not contain mutant producer and/or target cell(s). This step is conveniently achieved by Fluorescence-Activated Droplet Sorting (FADS), and in such embodiments the producer and/or target cells are fluorescently labelled.

The incubation step (d) is carried out for a period and under conditions selected by reference to the nature of the producer and target cells selected. Thus, this step may comprise maintaining the microdroplet library at a temperature of 15° C.-95° C. for at least 1 hour. In some embodiments, the microdroplet library is maintained at a temperature of: (a) 15° C.-42° C.; (b) 20° C.-40° C.; (c) 20° C.-37° C.; (d) 20° C.-30° C.; or (e) about 25° C.; (f) 40° C.-60° C.; (g) 60° C.-80° C.; or (h) 80° C.-98° C.

The incubation step (d) may comprise maintaining the microdroplet library at said temperature for about 2, 4, 6, 12, 24 or 48 hours, or for up to 7 days, for example for 1, 2, 3, 4, 5, 6 or 7 days. In other embodiments, the incubation step (d) comprises maintaining the microdroplet library at said temperature for up to 2 weeks, for example for 1 week or 2 weeks.

The screening step (e) may comprises eliminating microdroplets which contain target cells. This is conveniently achieved by FADS, in which case the target cells may be fluorescently labelled. In this way, droplets containing only mutant producer cells which have outgrown or overgrown to extinction the target cells (or producer cells in co-culture with resistant producer cell mutants) can be isolated by sorting, thereby greatly enriching for mutant producer cells which elaborate cytotoxic agents against the target cells.

The method may further comprise a potency analysis step carried out during incubation step (d), whereby the relative growth rate in co-culture of mutant producer cells and target cells is determined. This may comprise sampling for microdroplets which contain only mutant producer cells by FADS during the incubation step, for example at different time points. Thus, two or more successive rounds of FADS selection may be carried out during incubation in order to recover different classes of producer mutants based on the potency of the cytotoxic agent produced. In such embodiments, target cells may be fluorescently labelled and microdroplets which contain target cells are subjected to continued incubation.

Thus, the screening step (e) preferably comprises FADS sorting for microdroplets in which target cells have been outgrown or overgrown to extinction by mutant producer cells.

The producer prokaryotic species may be selected from archaea, for example selected from the phyla: (a) Crenarchaeota; (b) Euryarchaeota; (c) Korarchaeota; (d) Nanoarchaeota and (e) Thaumarchaeota, for example Haloferax volcanii or Sulfolobus spp. Alternatively, the producer prokaryotic species may be selected from bacteria, for example selected from: (a) actinomycetes; (b) Pseudomonas spp., and (c) Bacillus spp.

In preferred embodiments, the producer bacterial species is selected from Streptomyces spp., for example selected from: (a) Streptomyces coelicolor, (b) Streptomyces lividans; (c) Streptomyces venezuealae; (d) Streptomyces griseus; (e) Streptomyces avermetilis; and (f) Streptomyces bingchenggensis;

The target cell may be a bacterial or eukaryotic cell. For example, the target cell may be: (a) fungal; (b) mammalian; (c) a higher plant cell; (d) protozoal; (e) a helminth cell; (f) algal; or (h) an invertebrate cell.

In some embodiments, the target cell is a cancer cell, for example a human cancer cell.

In other embodiments, the target cell is a pathogenic bacterium.

In a second aspect, the invention provides a method of identifying a cytotoxic agent comprising screening mutant bacteria to identify producers of a cytotoxic agent active against a target cell according to a method as defined above.

In a third aspect, the invention provides a process for producing a cytotoxic agent comprising the method of the second aspect of the invention as defined above. Here, the process may further comprise synthesising or isolating said cytotoxic agent from the mutant bacteria, and may optionally further comprise mixing the synthesised or isolated cytotoxic agent with a pharmaceutically acceptable excipient to produce a pharmaceutical composition.

Other aspects and preferred embodiments of the invention are defined and described in the other claims set out below.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term gene is a term describing a hereditary unit consisting of a sequence of DNA that occupies a specific location on a chromosome or plasmid and determines a particular characteristic in an organism. A gene may determine a characteristic of an organism by specifying a polypeptide chain that forms a protein or part of a protein (structural gene); or encode an RNA molecule; or regulate the operation of other genes or repress such operation; or affect phenotype by some other as yet undefined mechanism.

The terms genomic DNA is a term of art used herein to define chromosomal DNA as distinct from extrachromosomally-maintained plasmid DNA.

The term genome is a term of art used herein to define the entire genetic complement of an organism, and so includes chromosomal, plasmid, prophage and any other DNA.

The term Gram-positive bacterium is a term of art defining a particular class of bacteria that are grouped together on the basis of certain cell wall staining characteristics.

The term low G+C Gram-positive bacterium is a term of art defining a particular subclass class of evolutionarily related bacteria within the Gram-positives on the basis of the composition of the bases in the DNA. The subclass includes Streptococcus spp., Staphylococcus spp., Listeria spp., Bacillus spp., Clostridium spp., Enterococcus spp. and Lactobacillus spp.).

The term high G+C Gram-positive bacterium is a term of art defining a particular subclass class of evolutionarily related bacteria within the Gram-positives on the basis of the composition of the bases in the DNA. The subclass includes actinomycetes (actinobacteria) including Actinomyces spp., Arthrobacter spp., Corynebacterium spp., Frankia spp., Micrococcus spp., Micromonospora spp., Mycobacterium spp., Nocardia spp., Propionibacterium spp. and Streptomyces spp.

The term Gram-negative bacterium is a term of art defining a particular class of bacteria that are grouped together on the basis of certain cell wall staining characteristics. Examples of Gram-negative bacterial genera include Klebsiella, Acinetobacter, Escherichia, Pseudomonas, Enterobacter and Neisseria.

The term “monodisperse” as applied to the microdroplets means any emulsion showing a coefficient of particle size dispersion, c, of not more than 1.0, not more than 0.5, and preferably not more than 0.3. Said coefficient E is defined by the following equation:

ε=(⁹⁰Dₚ−¹⁰Dₚ)/⁵⁰Dₚ (1)

where ¹⁰Dₚ, ⁵⁰Dₚ and ⁹⁰Dₚ are the particle sizes when the cumulative frequencies estimated from a relative cumulative particle size distribution curve for the emulsion are 10%, 50% and 90%, respectively. The case where ε=0 means an ideal state in which emulsion particles show no particle size scattering at all.

As used herein, the term “insertion rate” as applied to transposon insertion, is used to indicate the density of Tn_{A} insertion at the level of the mutant pool as a whole, with one Tn_{A} insertion in each bacterium. It will also be understood that lethal Tn_{A} insertion events, such as those arising from insertional inactivation of an essential gene, will not be represented by viable members of the mutant pool. Thus, the insertion rates specified herein apply to non-essential regions of the DNA.

As used herein, the term “cytotoxic agent” defines any compound (e.g. a metabolite, protein, peptide or other biopolymer) which acts to kill, or to prevent or restrict the growth or biological activity of, a target cell.

Producer Prokaryotic Cells

Any prokaryotic cell may be used according to the invention, including archaeal and bacterial cells.

Archaeal cells may be selected from the phyla: (a) Crenarchaeota; (b) Euryarchaeota; (c) Korarchaeota; (d) Nanoarchaeota and (e) Thaumarchaeota.

Exemplary, archaeal genera include Acidianus, Acidilobus, Acidococcus, Aciduliprofundum, Aeropyrum, Archaeoglobus, Bacilloviridae, Caldisphaera, Caldivirga, Caldococus, Cenarchaeum, Desulfurococcus, Ferroglobus, Ferroplasma, Geogemma, Geoglobus, Haladaptaus, Halalkalicoccus, Haloalcalophilium, Haloarcula, Halobacterium, Halobaculum, Halobiforma, Halococcus, Haloferax, Halogeometricum, Halomicrobium, Halopiger, Haloplanus, Haloquadratum, Halorhabdus, Halorubrum, Halosarcina, Halosimplex, Halostagnicola, Haloterrigena, Halovivax, Hyperthermus, lgnicoccus, lgnisphaera, Metallosphaera, Methanimicrococcus, Methanobacterium, Methanobrevibacter, Methanocalculus, Methantxaldococcus, Methanocella, Methanococcoides, Methanococcus, Methanocorpusculum, Methanoculleus, Methanofollis, Methanogenium, Methanohalobium, Methanohalophilus, Methanolacinia, Methanolobus, Methanomethylovorans, Methanomicrobium, Methanoplanus, Methanopyrus, Methanoregula, Methanosaeta, Methanosalsum, Methanosarcina, Methanosphaera, Melthanospirillum, Methanothermobacter, Methanothermococcus, Methanothermus, Methanothrix, Methanotorris, Nanoarchaeum, Natrialba, Natrinema, Natronobacterium, Natronococcus, Natronolimnobius, Natronomonas, Natronorubrum, Nitracopumilus, Palaeococcus, Picrophilus, Pyrobaculum, Pyrococcus, Pyrodictium, Pyrolobus, Staphylothermus, Stetteria, Stygiolobus, Sulfolobus, Sulfophobococcus, Sulfurisphaera, Thermocladium, Thermococcus, Thermodiscus, Thermofilum, Thermoplasma, Thermoproteus, Thermosphaera and Vulcanisaeta.

Exemplary archaeal species include: Aeropyrum pernix, Archaeglobus fulgidus, Archaeoglobus fulgidus, Desulforcoccus species TOK, Methanobacterium thermoantorophicum, Methanococcus jannaschii, Pyrobaculum aerophilum, Pyrobaculum calidifontis, Pyrobaculum islandicum, Pyrococcus abyssi, Pyrococcus GB-D, Pyrococcus glycovorans, Pyrococcus horikoshii, Pyrococcus spp. GE23, Pyrococcus spp. ST700, Pyrococcus woesii, Pyrodictium occultum, Sulfolobus acidocaldarium, Sulfolobus solataricus, Sulfolobus tokodalii, Thermococcus aggregans, Thermococcus barossii, Thermococcus celer, Thermococcus fumicolans, Thermococcus gorgonarius, Thermococcus hydrothermalis, Thermococcus onnurineus NA1, Thermococcus pacificus, Thermococcus profundus, Thermococcus siculi, Thermococcus spp. GE8, Thermococcus spp. JDF-3, Thermococcus spp. TY. Thermococcus thioreducens, Thermococcus zilligti, Thermoplasma acidophilum, Thermoplasma volcanium, Acidianus hospitalis, Acidilobus sacharovorans, Aciduliprofundum boonei, Aeropyrum pernix, Archaeoglobus fulgidus, Archaeoglobus profundus, Archaeoglobus veneficus, Caldivirga maquilingensis, Candidatus Korarchaeum cryptofilum, Candidatus Methanoregula boonei, Candidatus Nitrosoarchaeum limnia, Cenarchaeum symbiosum, Desulfurococcus kamchatkensis, Ferroglobus placidus, Ferroplasma acidarmanus, Halalkalicoccus jeotgali, Haloarcula hispanica, Holaoarcula marismortui, Halobacterium salinarum, Halobacterium species, Halobiforma lucisalsi, Haloferax volvanii, Halogeometricum borinquense, Halomicrobium mukohataei, halophilic archaceon sp. DL31, Halopiger xanaduensis, Haloquadratum walsbyi, Halorhabdus tiamatea, Halorhabdus utahensis, Halorubrum lacusprofundi, Haloterrigena turkmenica, Hyperthermus butylicus, Igniococcus hospitalis, lgnisphaera aggregans, Metallosphaera cuprina, Metallosphaera sedula, Methanobacterium sp. AL-21, Methanobacterium sp. SWAN-1, Methanobacterium thermoautrophicum, Methanobrevibacter ruminantium, Methanobrevibacter smithii, Methanocaldococcus fervens, Methanocaldococcus infernus, Methanocaldococcus jannaschii, Methanocaldococcus sp. FS406-22, Methanocaldococcus vulcanius, Methanocella conradii, Methanocella paludicola, Methanocella sp. Rice Cluster I (RC-I). Methanococcoides burtonii, Methanococcus aeolicus, Methanococcus maripaludis, Methanococcus vannielii, Methanococcus voltae, Methanocorpusculum labreantum, Methanoculleus marisnigri, Methanohalobium evestigatum, Methanohalophilus mahii, Methanoplanus petrolearius, Methanopyrus kandleri, Methanosaeta concilii, Methanosaeta harundinacea, Methanosaeta thermophila, Methanosalsum zhilinae, Methanosarcina acetivorans, Methanosarcina barkeri, Methanosarcina mazei, Methanosphaera stadtmanae, Methanosphaerula palustris, Methanospiriullum hungatei, Mathanothermobacter marburgensis, Methanothermococcus okinawensis, Methanothermus fervidus, Methanotorris igneus, Nanoarchaeum equitans, Natrialba asiatica, Natrialba magadii, Natronomonas pharaonis, Nitrosopumilus maritimus, Picrophilus torridus, Pyrobaculum aerophilum, Pyrobaculum arsenaticum, Pyrobaculum calidifontis, Pyrobaculum islandicum, Pyrobaculum sp. 1860, Pyrococcus abyssi, Pyrococcus furiosus, Pyrococcus horikoshii, Pyrococcus sp. NA42, Pyrococcus yayanosii, Pyrolobus fumarii, Staphylothermus hellenicus, Staphylothermus marinus, Sulfolobus acidocaldirius, Sulfolobus islandicus, Sulfolobus solfataricus, Sulfolobus tokodaii, Thermococcus barophilus, Thermococcus gammatolerans, Thermococcus kodakaraensis, Thermococcus litoralis, Thermococcus onnurineus, Thermococcus sibiricus, Thermococcus sp. 4557, Thermococcus sp. AM4, Thermofilum pendens, Thermoplasma acidophilum, Thermoplasma volcanium, Thermoproteus neutrophilus, Thermoproteus tenax, Thermoproteus uzoniensis, Thermosphaera aggregans, Vulcanisaeta distributa, and Vulcanisaeta moutnovskia.

Particular examples of archaeal cells useful as producer cells according to the invention include Haloferax volcanii and Sulfolobus spp.

Bacterial cells may be selected from the phylum Actinobacteria, for example from the following families: Actinomycetaceae; Propionibacteriaceae; Frankiaceae; Micrococcaceae; Micromonosporaceae; Streptomycetaceae; Mycobacteriaceae; Corynebacteriaceae; Pseudonocardiaceae and Nocardiaceae.

Thus, in some embodiments, the producer prokaryotic cell is selected from Saccaropolyspora spp., for example Saccaropolyspora erythrea. In other embodiments, the producer prokaryotic cell is selected from Kutzneria spp., for example Kutzneria albida. In yet other embodiments, the producer prokaryotic cell is selected from Mycobacterium spp., for example Mycobacterium marinum.

In preferred embodiments, the producer prokaryotic cell is selected from Streptomyces spp. The Streptomyces genus comprises more than 500 species, any of which may be used as producer strains according to the invention. Thus, the producer strain may be selected from any of the following species: S. ambofaciens, S. achromogenes, S. anulatus, S. avermetilis, S. coelicolor, S. clavuligerus, S. felleus, S. ferralitis, S. filamentosus, S. griseus, S. hygroscopicus, S. iysosuperficus, S. lividans, S. noursei, S. scabies, S. somaliensis, S. thermoviolaceus, S. venezuelae and S. violaceoruber.

In some embodiments, the producer prokaryotic cell is selected from: (a) Streptomyces coelicolor, (b) Streptomyces lividans; (c) Streptomyces venezuealae; (d) Streptomyces griseus; (e) Streptomyces avermetilis; and (f) Streptomyces bingchenggensis.

In other embodiments, the producer prokaryotic cell is selected from Micromonospora spp., which genus comprises several species, any of which may be used as producer strains according to the invention. Thus, the producer cell may be selected from any of the following species: M. aurantiaca, M. carbonacea, M. chalcea, M. chersina, M. citrea, M. coerulea, M. echinaurantiaca, M. echinofusca, M. echinospora, M. fulviviridis, M. gallica, M. halophytica, M. inositola, M. inyonensis, M. nigra, M. olivasterospora, M. pallida, M. peucetia, M. purpureochromogenes, M. rosaria, M. sagamiensis and M. viridifaciens.

The bacterial cells may also be selected from the phylum Firmicutes, for example from the following classes: Bacilli, Clostridia and Mollicutes.

Exemplary Bacilli include those selected from any of the following families: Alicyclobacillaceae; Bacillaceae; Caryophanaceae; Listeriaceae; Paenibacillaceae; Planococcaceae; Sporolactobacillaceae; Staphylococcaceae; Thermoactinomycetaceae and Turicibacteraceae; Exemplary Clostridia include those selected from any of the following families: Acidaminococcaceae; Clostridiaceae; Eubacteriaceae; Heliobacteriaceae; Lachnospiraceae; Peptococcaceae; Peptostreptococcaceae and Syntrophomonadaceae.

In some embodiments, the producer prokaryotic cell is selected from Bacillus spp. and Clostridia spp., for example Bacillus amyloliquefaciens subsp. Plantarum;

Bacterial cells may also be selected from the family Pseudomonadaceae, for example members of the genus Pseudomonas.

Target Cells for Use in the Methods of the Invention

Bacterial Target Cells

The target cells for use according to the invention may be bacterial cells. In such embodiments, the bacteria may be selected from: (a) Gram-positive, Gram-negative and/or Gram-variable bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, target cells for use according to the invention may be selected from bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorferi); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundii); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylon); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morganii); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. serotypes. Typhi and Typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. Y. pestis).

The target cells for use according to the invention may be selected from high G+C Gram-positive bacteria and in low G+C Gram-positive bacteria.

Pathogenic Bacteria as Target Cells

Human or animal bacterial pathogens include such bacteria as Legionella spp., Listeria spp., Pseudomonas spp., Salmonella spp., Klebsiella spp., Hafnia spp, Haemophilus spp., Proteus spp., Serratia spp., Shigella spp., Vibrio spp., Bacillus spp., Campylobacter spp., Yersinia spp. Clostridium spp., Enterococcus spp., Neisseria spp., Streptococcus spp., Staphylococcus spp., Mycobacterium spp., Enterobacter spp.

Pathogenic Fungal Cells

These include yeasts, e.g. Candida species including C. albicans, C krusei and C tropicalis, and filamentous fungi such as Aspergillus spp. and Penicillium spp. and dermatophytes such as Trichophyton spp.

Plant Pathogens

The target cells for use according to the invention may be plant pathogens, for example Pseudomonas spp., Xylella spp., Ralstonia spp., Xanthomonas spp., Erwinia spp., Fusarium spp., Phytophthora spp., Botrytis spp., Leptosphaeria spp., powdery mildews (Ascomycota) and rusts (Basidiomycota).

Mutant Producer Cell Pools

The methods of the invention involve generating a pool of mutant prokaryotic cells by transposon mutagenesis. The size of the mutant pool affects the resolution of the method: as the pool size increases, more and more different genes with Tn_{A} insertions will be represented (and so effectively assayed). As the pool size decreases, the resolution of the method reduces, genes will be less effectively assayed, and more and more genes will not be assayed at all.

Ideally, the mutant pool generated in the methods of the invention is comprehensive, in the sense that insertions into every (non-essential) gene are represented. The number of Tn_{A} insertion mutants (i.e. the mutant pool size) required to achieve this depends on various factors, including: (a) the size of the prokaryotic genome; (b) the average size of the genes; and (c) any Tn_{A} insertion site bias.

With regard to the latter, some areas of the genome attract a low frequency of insertion (especially GC-rich regions). Thus, insertion frequencies and pool sizes large enough to ensure insertions into insertion-refractory regions are preferred.

In general, a minimum insertion rate of one transposon per 25 bp is required to achieve a comprehensive pool/library, which typically entails a minimum pool size for prokaryotic cells having a genome size of 4 to 7 Mb of 0.5×10⁵ to 1×10⁵, for example 5×10⁵, preferably at least about 1×10⁶ mutants. In many cases, 1×10⁶ mutants will allow identification of ˜300,000 different insertion sites and correspond to 1 transposon insertion every 13 to 23 bp (or about 40-70 different insertion sites per gene).

However, the methods of the invention do not necessarily require a comprehensive mutant pool (in the sense defined above) in order to generate useful hits. Rather, pool sizes less than the ideal comprehensive pool may be used, provided that a reduction in resolution (and attendant failure to assay certain genes) can be tolerated. This may be the case, for example, where the method is designed to be run iteratively until a hit is identified: in such embodiments the effective pool size grows with each iteration of the method.

Transposon Mutagenesis

Transposons, sometimes called transposable elements, are polynucleotides capable of inserting copies of themselves into other polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ends of RNA transcripts with 5′ ends often truncated.

Transposomes are transposase-transposon complexes wherein the transposon does not encode transposase. Thus, once inserted the transposon is stable. Preferably, in order to ensure mutant pool stability, the transposon does not encode transposase and is provided in the form of a transposome (i.e. as a complex with transposase enzyme), as described below.

As used herein, the term “activating transposon” (hereinafter abbreviated “Tn_{A}”) defines a transposon which comprises a promoter such that transposon insertion increases the transcription of a gene at or near the insertion site. Examples of such transposons are described in Troeschel et al. (2010) Methods Mol Biol. 668:117-39 and Kim et al. (2008) Curr Microbiol. 57(4): 391-394.

The activating transposon/transposome can be introduced into the prokaryotic genome (including chromosomal and/or plasmid DNA) by any of a wide variety of standard procedures which are well-known to those skilled in the art. For example, Tn_{A} transposomes can be introduced by electroporation (or any other suitable transformation method).

Preferably, the transformation method generates 1×10³ to 5×10³ transformants/ng DNA, and such transformation efficiencies are generally achievable using electroporation.

Alternatively, transposon mutagenesis using Tn_{A} may be performed in vitro and recombinant molecules transformed/transfected into the prokaryotic cells. In such embodiments, transposomes can be prepared according to a standard protocol by mixing commercially available transposase enzyme with the transposon DNA fragment. The resulting transposomes are then mixed with extra-chromosomal DNA of interest to allow transposition, then the DNA is introduced into a host bacterial strain using electrotransformation to generate a pool of extra-chromosmal DNA transposon mutants.

In embodiments where mutagenesis is performed in vitro, it is possible to mix transposomes with genomic DNA in vitro and then introduce the mutagenized DNA (optionally, after fragmentation and/or circularization) into the host prokaryotic cell (e.g. by electroporation) whereupon endogenous recombination machinery incorporates it into the genome. Such an approach may be particularly useful in the case of prokaryotes which are naturally competent (e.g. Acinetobacter spp.) and/or can incorporate DNA via homologous crossover (e.g. double crossover) recombination events.

Activating Transposons for Use in the Methods of the Invention

Any suitable activating transposon may be used in the methods of the invention. Suitable transposons include those based on Tn3 and the Tn3-like (Class II) transposons including γδ (Tn 1000), Tn501, Tn2501, Tn21, Tn917 and their relatives. Also Tn 10, Tn5, TnphoA, Tn903, TN5096, Tn5099, Tn4556, UC8592, IS493, bacteriophage Mu and related transposable bacteriophages. A variety of suitable transposons are also available commercially, including for example the EZ-Tn5™<R6Kγori/KAN-2> transposon.

Preferred transposons are those which carry antibiotic resistance genes although any selectable marker can be used including auxotrophic complimentation (which may be useful in identifying mutants which carry a transposon), including Tn5, Tn 10 and TnphoA. For example, Tn 10 carries a tetracycline resistance gene between its IS elements while Tn5 carries genes encoding polypeptides conferring resistance to kanamycin, streptomycin and bleomycin. Other suitable resistance genes include those including neomycin, apramycin, thiostrepton and chloramphenicol acetyltransferase (conferring resistance to chloramphenicol).

It is of course possible to generate new transposons by inserting different combinations of antibiotic resistance genes between IS elements, or by inserting combinations of antibiotic resistance genes between transposon mosaic ends (preferred), or by altering the polynucleotide sequence of the transposon, for example by making a redundant base substitution or any other type of base substitution that does not affect the transposition or the antibiotic resistance characteristics of the transposon, in the coding region of an antibiotic resistance gene or elsewhere in the transposon. Such transposons are included within the scope of the invention.

In many embodiments, a single transposon is used to generate the mutant pool. However, as explained above, the number of Tn insertion mutants (i.e. the mutant pool size) required to achieve a comprehensive pool or library depends inter alia on any Tn insertion site bias. Thus, in cases where the transposon insertion site bias occurs, two or more different transposons may be used in order to reduce or eliminate insertion site bias. For example, a combination of two different transposons based on Tn5 and Tn 10 may be employed.

Promoters for Use in Activating Transposons

The nature of the promoter present in the Tn_{A} is dependent on the nature of the transposon and the ultimate prokaryotic host. Generally, an efficient, outward-oriented promoter which drives constitutive and/or high level transcription of DNA near or adjacent to the insertion site is chosen.

The promoter may include: (a) a Pribnow box (−10 element); (b) a −35 element and/or (c) an UP element.

For example, the lac promoter can be used with the EZ-Tn5™<R6Kγori/KAN-2> transposon, and such constructs are suitable for assay of e.g. Escherichia coli, Enterobacter spp. and other members of the family Enterobacteriaceae such as Klebsiella spp. Other suitable promoters include: rpIJ (large ribosomal subunit protein; moderate strength promoter); tac (artificial lac/trp hybrid; strong promoter) and rrnB (ribosomal RNA gene promoter; very strong promoter).

Suitable promoters can also be engineered or selected as described in Rhodius et al. (2011) Nucleic Acids Research: 1-18 and Zhao et al. (2013) ACS Synth. Biol. 2 (11): 662-669. The rapid application of next generation sequencing to RNA-seq is now providing a wealth of high-resolution information of transcript start sites at a genomic level, which greatly simplifies the identification of promoter sequences in any given prokaryote. This permits the construction of descriptive promoter models for entire genomes. RNA-seq also provides quantitative information on transcript abundance and hence promoter strength, which enables the construction of promoter strength models that can then be used for predictive promoter strength rankings (see Rhodius et al. (2011) Nucleic Acids Research: 1-18).

Thus, real-time PCR and RNA-seq permit the rapid identification of strong constitutive promoters from the upstream regions of the housekeeping genes in the selected producer cell.

Suitable promoters can also be identified by assay. For example, a series of plasmids can be constructed to test promoter strength empirically. Briefly, the promoter to be tested is placed upstream of an antibiotic resistance gene and then transformed into the relevant bacteria. General cloning assembly and plasmid amplification can be carried out in E. coli (facilitated by the ampicillin resistance gene and the pBR322 ori) and the activity of the promoter in the target bacterium can then be assayed by generating a killing curve with the relevant antibiotic—a very high level promoter gives more antibiotic resistance expression and therefore survival at a higher antibiotic concentration. The plasmid series is designed to be modular so that the origin of replication, resistance gene(s) and promoter can be easily switched.

Suitable promoters for use with Actinobacteria in general (and Streptomyces spp. in particular) include the actinobacterial gapdh and rpsL promoters described in Zhao et al. (2013) ACS Synth. Biol. 2 (11): 662-669.

In circumstances where multiple promoters of different strengths are to be used (see below for more details in this regard), then gapdh from Eggerthella lenta and rpsL from Cellulomonas flavigina may be used as the basis for very strong promoters, gapdh and rpsL from S. griseus may be used as the basis for medium strength promoters while rpoA and rpoB from S. griseus may be used as the basis for low strength promoters (see Shao et al. (2013) ACS Synth. Biol. 2 (11): 662-669).

Other suitable promoters include ermE*, a mutated variant of the promoter of the erythromycin resistance gene from Saccharopolyspora erythraea (see e.g. Wagner et al. (2009) J. Biotechnol. 142: 200-204).

Suitable promoters for use with Actinobacteria may be identified and/or engineered as described in Seghezzi et al. (2011) Applied Microbiology and Biotechnology 90(2): 615-623, where the use of randomised −10 and −35 boxes to identify important sequences for expression levels is described. Another approach is described in Wang et al. (2013) Applied and Environmental Microbiology 79(14): 4484-4492.

Suitable promoters for use with Bacillus spp. May be based on the many different promoters described for the model organism Bacillus subtilis, including for example the P43, amyE and aprE promoters from B. subtilis (see e.g. Kim et al. (2008) Biotechnology and Bioprocess Engineering 13(3) 313-318).

Use of Multiple Tn_{A}/Multiple Promoters

In some embodiments of the invention, the prokaryotic genome is probed with a mixture of different activating transposons which have outward facing promoters of different strengths. In some circumstances, a broader range of genes involved in antibiotic resistance and/or sensitivity are recovered if a mixture of activating transposons with at least three different promoters of progressively decreasing strength are employed to generate the mutant pool.

In such circumstances, the use of a plurality of activating transposons with promoters of varying strength ensures that transposon insertions occur substantially in all non-essential genes are represented in the initial mutant pool, since transposon insertion can now result in gene activation to yield an appropriate level of transcription (neither too high, nor too low).

In such embodiments, a wide variety of promoters may be used provided that at least three different promoters are used wherein the relative strength of said promoters is: Tn_{A}P1>Tn_{A}P2>Tn_{A}P3; such that transposon insertion into prokaryotic DNA generates a pool of mutant cells containing members in which one or more genes are transcribed from Tn_{A}P1, one or more genes are transcribed from Tn_{A}P2 and one or more genes are transcribed from Tn_{A}P3.

Preferably, Tn_{A}P1 is a strong promoter, Tn_{A}P2 a medium-strength promoter and Tn_{A}P3 a weak promoter in the mutagenized prokaryotic host cells under the conditions used for incubation and culture of the mutant pool in the presence of the target cells. In some embodiments, the relative transcription initiation rate of Tn_{A}P1 is at least 3 times, at least 100 times, at least 1000 times or at least 10000 times higher than that of Tn_{A}P3 under these conditions.

Each promoter typically includes: (a) a Pribnow box (−10 element); (b) a −35 element and (c) an UP element. Those skilled in the art are able to readily identify promoters having the required relative strengths by sequence analysis and/or in vitro or in vivo assays using expression constructs.

Suitable promoters include the E. coli rplJ (large ribosomal subunit protein; moderate strength promoter); tac (artificial lac/trp hybrid; strong promoter) and rrnB (ribosomal RNA gene promoter; very strong promoter) promoters.

As used herein, the terms P_{rplJ} and P_{rrnB} specifically refer to the E. coli promoters for the 50S ribosomal subunit protein L10 and 16S ribosomal RNA genes, respectively. Orthologues of these (and other) E. coli promoters from other Gram-negative bacteria can also be used, including in particular the orthologous Pseudomonas aeruginosa or Acinetobacter baumannii promoters.

For example, the orthologous Acinetobacter baumannii gene corresponding to the E. coli rrnB P_{rrnB} has the gene symbol A1S_r12 and encodes the Acinetobacter baumannii 16S ribosomal RNA gene, so that the corresponding orthologous promoter is herein designated P_{(A1S}_{_}ᵣ₁₂₎. Thus, when the method is applied to Acinetobacter baumannii, Tn_{P1} may be P_{(A1S}_{_}ᵣ₁₂₎.

Similarly, when the method of the invention is applied to Pseudomonas aeruginosa, Tn_{P2} may be the 16S ribosomal RNA gene promoter from P. aeruginosa (i.e. Ps.P_{rrnB}) while Tn_{P3} may be selected from the rpsJ (small (30S) ribosomal subunit S10 protein) gene promoter from P. aeruginosa (i.e. Ps.P_{rps,J}) and the E. coli P_{rrnB}.

Effects of Tn_{A} Insertion: Generation of Mutant Producer Cells Expressing Cytotoxic Compounds

The use of transposon mutagenesis with an activating transposon (Tn_{A}) according to the methods of the invention is capable of producing an extremely diverse range of phenotypes, since the effect of insertion of the Tn_{A} can vary from total loss of function, decreased activity, to varying degrees of increased activity, with change of function (and even gain of function) also being possible.

This follows from the fact that the effect of insertion of the Tn_{A} into the DNA of the prokaryotic producer cell is sequence context dependent. Insertion into the coding sequence of a structural gene may result in insertional inactivation (and so complete loss of function), while insertion upstream of a gene or operon can result in Tn_{A}P driving increased transcription and so lead to expression of that gene or operon (with the extent of overexpression being dependent on subtle positional/polar effects).

A further layer of complexity is introduced by the fact that Tn_{A} insertion may result in transcripts from both sense and anti-sense strands, resulting in the production of antisense transcripts arising from TnAP driving transcription of the non-coding strand of the DNA of the mutant prokaryotic producer cell. Such antisense transcripts may suppress or activate gene expression (for example they may suppress expression of genes by binding to complementary mRNA encoded by the corresponding coding (sense) strand of the DNA of said prokaryotic cell), or may activate gene transcription by suppressing expression of genes encoding gene repressors.

Some of the mechanisms by which transposon mutagenesis with an activating transposon (Tn_{A}) according to the methods of the invention give rise to mutants expressing cytotoxic agents are described in detail below:

Activation of Secondary Metabolic Processes

Many cytotoxic compounds are the products of secondary metabolic pathways and as such are subject to regulatory mechanisms that serve to maintain the primacy of the primary metabolic pathways required for growth. These mechanisms can limit the production of cytotoxic products to levels which are undetectable and/or inactive in screens based on screens for activity against co-cultured target cells.

The use of transposon mutagenesis with an activating transposon (Tn_{A}) according to the methods of the invention may result in Tn_{A} insertion into cellular DNA of the producer prokaryotic species which:
(a) occurs up-stream of an activator of a cryptic cytotoxic agent gene or operon, whereat Tn_{A}P drives increased transcription of the activator which acts in trans to increase expression of the cryptic cytotoxic agent gene or operon; or
(b) insertionally inactivates a repressor of a cryptic cytotoxic agent gene or operon, thereby increasing expression of the cryptic cytotoxic agent gene or operon;

In either case, mutants in which existing control of secondary metabolic pathways responsible for the production of cytotoxic agents is effectively overridden are generated.

Overexpression and Inactivation of Structural Genes

In this context, structural genes are considered to be those coding for any RNA or protein product not associated with regulation.

Structural gene overexpression may increase production of cytotoxic compounds of interest and Tn_{A} insertion up-stream of an enzyme involved in cytotoxic agent synthesis, whereat Tn_{A}P drives increased transcription and so leads to increased expression of said enzyme, may therefore result in the generation of an important class of mutants of interest. In a similar fashion, Tn_{A} insertion up-stream of an enzyme which produces an cytotoxic agent as a side activity, whereat Tn_{A}P drives increased transcription and so leads to increased expression of said enzyme, thereby increasing its side activity and increasing levels of the cytotoxic agent;

Structural gene inactivation may also increase production of cytotoxic compounds of interest. This is particularly applicable when applied to systems in which one natural product is biosynthetically transformed to another, less useful, compound. Tn_{A} insertion into cellular DNA of the producer prokaryotic species which insertionally inactivates an enzyme for which an cytotoxic agent is a substrate, so increasing levels of the cytotoxic agent, may therefore result in the generation of mutants of interest.

Overexpression of Export Genes

Natural product biosynthesis is often subjected to negative feedback regulation. Clearance from the cell of compounds mediating negative feedback regulation by export proteins or efflux systems can therefore result in the production of cytotoxic compounds of interest. For example, doxorubicin production in Streptomyces peucetius can be enhanced over two-fold by overexpression of the export genes drrA and drrB.

Thus, Tn_{A} insertion up-stream of an export gene involved in clearance of compounds mediating negative feedback regulation of cytotoxic agent synthesis, whereat Tn_{A}P drives increased transcription and so leads to increased expression of said export gene, may therefore result in the generation of another important class of mutants of interest.

Precursor Supply

Bottlenecks in biosynthetic pathways can restrict the synthesis of natural products and may be associated with limited provision of key precursors by primary metabolic pathways. Such limitations can be overcome by upregulation of gene or operons that code for enzymes associated with such bottlenecks: increased enzyme levels translate to diminished bottleneck effects and hence improved synthesis of the cytotoxic compounds of interest.

Thus, Tn_{A} insertion up-stream of an enzyme associated with such bottlenecks in cytotoxic agent synthesis, whereat Tn_{A}P drives increased transcription and so leads to increased expression of said enzyme, may therefore result in the generation of a yet further class of mutants of interest.

Activation of Cryptic Genes

Many cytotoxic compounds of interest are the products of secondary metabolic processes which are silent under most growth conditions. Such process may be induced only during particular phases of growth, under certain growth conditions, during particular developmental stages (e.g. sporulation), induction by bacterial cytokines (e.g. as part of a quorum sensing system), stimulation by factors produced by other organisms, nutrient status, temperature, stress or other inter-cellular microbial regulators.

For example, recent genome sequencing projects with Streptomyces spp. have revealed that they possess many ‘cryptic’ antibiotic biosynthetic pathways; that is they have the genetic potential to produce many more antibiotics than previously realised. These cryptic pathways are not genetic relics but can be activated to direct production of new antibiotics.

The use of transposon mutagenesis with an activating transposon (Tn_{A}) according to the methods of the invention may result in Tn_{A} insertion into cellular DNA of the producer prokaryotic species which:
(a) occurs up-stream of a cryptic cytotoxic agent gene or operon, whereat Tn_{A}P drives increased transcription and so leads to expression of the cryptic cytotoxic agent gene or operon; or
(b) occurs up-stream of an activator of a cryptic cytotoxic agent gene or operon, whereat Tn_{A}P drives increased transcription of the activator which acts in trans to increase expression of the cryptic cytotoxic agent gene or operon; or
(c) insertionally inactivates a repressor of a cryptic cytotoxic agent gene or operon, thereby increasing expression of the cryptic cytotoxic agent gene or operon.

In each case, mutants in which cryptic metabolic pathways responsible for the production of cytotoxic agents are unveiled are generated.

Functional Domain Effects

Enzymes involved in the synthesis of cytotoxic agents may be comprised of multiple, functionally distinct, domains. Tn_{A} insertion into cellular DNA of the producer prokaryotic species which:
(a) insertionally inactivates one or more domains of a multi-domain enzyme, thereby altering its function such that it synthesises, directly or indirectly, an cytotoxic agent; or
(b) occurs up-stream of one or more domains of a multi-domain enzyme, whereat Tn_{A}P drives increased transcription of said one or more domains, thereby altering the function of said enzyme such that it synthesises, directly or indirectly, an cytotoxic agent;

may therefore result in the generation of a yet further class of mutants of interest.

Finally, the use of transposon mutagenesis with an activating transposon (Tn_{A}) according to the methods of the invention may result in Tn_{A} insertion into cellular DNA of the producer prokaryotic species which alters the coding repertoire of the prokaryotic producer cell such that an entirely novel cytotoxic agent is directly or indirectly expressed (i.e. the resultant mutation may be neomorphic).

Determining the Sequence Context of Tn_{A} Insertions

As explained above, the use of transposon mutagenesis with an activating transposon (Tn_{A}) according to the methods of the invention results in the production of a richly diverse mutant pool.

This aspect of the invention is complemented by the ability to establish the sequence context of transposon insertions associated with the production of cytotoxic agents of interest. This greatly facilitates the identification of the cytotoxic agent, the metabolic pathways by which it is synthesised in the cell as well as its mode of action (since the distribution of Tn_{A} insertions across the entire mutant pool may reveal the identity of resistance factors/mechanisms in co-cultured resistance mutants of the prokaryotic cells).

The sequence context is preferably determined by sequencing DNA adjacent or near (5′ and/or 3′) the Tn_{A} insertion site (e.g. by sequencing DNA which comprises Tn_{A}-genomic DNA junctions). Typically, bacterial DNA flanking or adjacent to one or both ends of the Tn_{A} is sequenced.

The length of adjacent DNA sequenced need not be extensive, and is preferably relatively short (for example, less than 200 base pairs).

Various methods can be used to determine the Tn_{A} insertion distribution using DNA sequencing: such methods have recently been dubbed Tn-seq procedures (van Opijnen et al. (2009) Nat. Methods 6: 767-772). For example, Tn-seq procedures include affinity purification of amplified Tn junctions (Gawronski et al. (2009) PNAS 106: 16422-16427); ligation of adaptors into genome sequences distal to the end of the transposon using a specialized restriction site (Goodman et al. (2009) Cell Host Microbe 6: 279-289; van Opijnen et al. (2009) Nat. Methods 6: 767-772); selective amplification (Langridge et al. (2009) Genome Research 19: 2308-2316) and the generation of single-stranded DNA circles bearing Tn junctions, which serve as templates for amplification and sequencing after elimination of genomic DNA by exonuclease digestion (Gallagher et al. (2011) mBio 2(1):e00315-10).

Any suitable high-throughput sequencing technique can be used, and there are many commercially available sequencing platforms that are suitable for use in the methods of the invention. Sequencing-by-synthesis (SBS)-based sequencing platforms are particularly suitable for use in the methods of the invention: for example, the Illumina™ system is generates millions of relatively short sequence reads (54, 75 or 100 bp) and is particularly preferred.

Other suitable techniques include methods based on reversible dye-terminators. Here, DNA molecules are first attached to primers on a slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labelled nucleotides then the dye along with the terminal 3′ blocker is chemically removed from the DNA, allowing a next cycle.

Other systems capable of short sequence reads include SOLiD™ and Ion Torrent technologies (both sold by Applied Biosystems™). SOLiD™ technology employs sequencing by ligation. Here, a pool of all possible oligonucleotides of a fixed length are labelled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion Torrent Systems Inc. have developed a system based on using standard sequencing chemistry, but with a novel, semiconductor based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

Functional Assessment of Mutants

Analysis of the sequence information described above may permit an assessment of the functional role of one or more cellular elements in the production and/or mode of action of the cytotoxic agent.

Suitable analytical techniques include bioinformatics, where the (full or partial) sequence of the genetic elements affected by Tn_{A} insertion is used to interrogate sequence databases containing information from the prokaryotic cell assayed and/or other species in order to identify genes (e.g. orthologous genes in other species) for which essential biochemical function(s) have already been assigned and/or which have been shown to be essential.

Suitable bioinformatics programs are well known to those skilled in the art and include the Basic Local Alignment Search Tool (BLAST) program (Altschul et al. (1990) J. Mol. Biol. 215: 403-410 and Altschul et al. (1997) Nucl. Acids Res. 25: 3389-3402). Suitable databases include, for example, EMBL, GENBANK, TIGR, EBI, SWISS-PROT and trEMBL.

Alternatively, or in addition, the (full or partial) sequence of the implicated genes/genetic elements is used to interrogate a sequence database containing information as to the identity of genes which has been previously constructed using the conventional Tn-seq methods described in the prior art (e.g. as described in Gawronski et al. (2009) PNAS 106: 16422-16427; Goodman et al. (2009) Cell Host Microbe 6: 279-289; van Opijnen et al. (2009) Nat. Methods 6: 767-772; Langridge et al. (2009) Genome Research 19: 2308-2316; Gallagher et al. (2011) mBio 2(1):e00315-10) and/or the techniques described in WO 01/07651 (the contents of which are hereby incorporated by reference).

The position of the inserted promoter can be assessed with respect to its contribution to increased transcription of relevant downstream DNA sequences. A mathematically/technically straightforward bioinformatics component of this technique permits recognition of the contribution of the inserted promoter sequence to transcription of the putative cytotoxic agent gene. Bioinformatics would allow the effects of transcriptional read through on genes downstream of the gene adjacent to the inserted transposon to be considered, where there is there no defined RNA transcription termination sequence.

Microdroplet Co-Encapsulation

The methods of the invention are suitable for high-throughput screening, since the methods involve compartmentalizing the screening assay in tiny volumes of growth medium in the form of discrete microdroplets. This permits each microdroplet to be treated as a separate culture vessel, permitting rapid screening of large numbers of individual liquid co-cultures using established microfluidic and/or cell-sorting methodologies.

Thus, the microdroplets of the invention function as individual, discrete culture vessels in which the producer and target cells can be co-cultured, analysed, manipulated, isolated and/or sorted.

Any suitable method may be employed for co-encapsulating the mutant producer cells and target cells in microdroplets according to the invention. For example, the mutant producer cells and target cells can be co-encapsulated in gel microdroplets according to the methods described in WO98/41869. Thus, gel droplets can be made using ionic or thermal gelling principles: typically, producer cells and target cells are mixed with a fluid precursor of the gel matrix. This is then solidified by polymerization, causing as little trauma to the cells as possible: for example, shocks arising from changes in temperature, osmotic pressure and pH should be minimized. The use of hydrogels is generally preferred, since they exhibit high water retention and porosity, permitting free diffusion of nutrients and waste products. Many such matrices are known in the art, including polysaccharides, such as alginates, carageenans, agarose, chitosan, cellulose, pectins and polyacrylamides.

Producer and target cells can be added to the gel matrix material to form a suspension and distributed evenly while the matrix is in liquid phase. Gel regions or gel droplets are formed by hardening of the matrix following the formation of small individual volumes of the gel matrix suspension droplets using established techniques. Suitable techniques include, for example, emulsification with oil, vortexing, sonication, homogenization, dropping using a syringe type apparatus, vibration of a nozzle attached to a reservoir of the suspension or atomization followed by electrostatic separation or cutting with rotating wires.

Emulsion Co-Encapsulation

Preferred according to the invention is co-encapsulation by emulsification. As described below, both single and double-phase emulsions may be used, including water-in-oil (W/O) type and water-in-oil-in water (W/O/W) type emulsions. In W/O emulsions, the dispersed phase may comprise microdroplets of aqueous growth medium suspended in a continuous oil phase. In W/O/W emulsions, the dispersed phase may comprise microdroplets of aqueous growth medium enveloped in an oil phase, which microdroplets are in turn suspended in a continuous aqueous phase. Such W/O/W emulsions may exhibit improved rheological properties over simple W/O emulsions: they may for example exhibit lower viscosity, permitting higher flow rates/lower running pressures when sorted using FADS and/or processed using microfluidic devices (see below).

Thus, in the simplest embodiments of the invention, co-encapsulation involves the formation of emulsions comprising microdroplets of liquid growth medium containing mutant producer and target cells dispersed in a carrier liquid as the continuous phase. Such single emulsions are of the W/O type, though it will be appreciated that any suitable liquid may be used as the continuous phase provided that it is immiscible with the growth medium selected for co-culture of the producer and target cells. Typically, however, the carrier liquid is an oil.

Depending inter alia on the nature of the continuous phase (e.g. the type of oil) and the droplet size, single emulsions of the type described above may have a viscosity that makes sorting of the microdroplets by certain microfluidic and/or cell sorting techniques difficult. For example, the viscosity may limit the flow rates achievable (e.g. to the range of 10-100 microdroplets per second using FADS—see below) and/or may require undesirably high operating pressures.

Double Emulsions

Particularly preferred according to the invention are double emulsions of the W/O/W type. Such emulsions comprise microdroplets containing an aqueous core of liquid aqueous growth medium containing mutant producer and target cells enveloped in a shell of immiscible liquid (typically an oil), these microdroplets being dispersed in an aqueous carrier liquid as the continuous phase.

Co-cultures encapsulated in this way can exhibit very low viscosities, and can therefore be sorted using e.g. FADS (see below) at much higher flow rates, permitting sorting of over 10,000 microdroplets per second (see e.g. Bernath et al. (2004) Analytical Biochemistry 325: 151-157).

Co-encapsulation in double emulsions may be achieved by any of a wide variety of techniques, using a wide range of suitable aqueous growth media, carrier oils and surfactants. Suitable techniques for making double emulsions (and for sorting them using FACS) is described, for example, in Bernath et al. (2004) Analytical Biochemistry 325: 151-157.

Surfactants for Use in Emulsion Co-Encapsulation

As explained herein, the microdroplets (and the corresponding microcultures) of the invention may comprise a single water-in-oil (W/O) or double water-in-oil-in-water (W/O/W) type emulsions, and in such embodiments one or more surfactants may be necessary to stabilize the emulsion.

The surfactant(s) and/or co-surfactant(s) are preferably incorporated into the W/O interface(s), so that in embodiments where single W/O type emulsions are used the surfactant(s) and or co-surfactant(s) may be present in at the interface of the aqueous growth medium microdroplets and the continuous (e.g., oil) phase. Similarly, where double W/O/W type emulsions are used for co-encapsulation according to the invention, the surfactant(s) and or co-surfactant(s) may be present at either or both of the interfaces of the aqueous core and the immiscible (e.g. oil) shell and the interface between the oil shell and the continuous aqueous phase.

A wide range of suitable surfactants are available, and those skilled in the art will be able to select an appropriate surfactant (and co-surfactant, if necessary) according to the selected screening parameters. For example, suitable surfactants are described in Bernath et al. (2004) Analytical Biochemistry 325: 151-157; Holtze and Weitz (2008) Lab Chip 8(10): 1632-1639; and Holtze et al. (2008) Lab Chip. 8(10):1632-1639.

The surfactant(s) are preferably biocompatible. For example, the surfactant(s) may be selected to be non-toxic to the mutant producer and target cells used in the screen). The selected surfactant(s) may also have good solubility for gases, which may be necessary for the growth and/or viability of the encapsulated cells.

Biocompatibility may be determined by any suitable assay, including assays based on tests for compatibility with a reference sensitive biochemical assay (such as in vitro translation) which serves as a surrogate for biocompatibility at the cellular level. For example, in vitro translation (IVT) of plasmid DNA encoding the enzyme 3-galactosidase with a fluorogenic substrate (fluorescein di-β-D-galactopyranoside (FDG)) may be used as an indicator of biocompatibility since a fluorescent product is formed when the encapsulated DNA, the molecules involved in transcription and translation, and the translated protein do not adsorb to the drop interface and the higher-order structure of the protein remains intact.

The surfactant(s) may also prevent the adsorption of biomolecules at the microdroplet interface. This may increase the sensitivity of the screen by ensuring that target cells are fully exposed to cytotoxic agents secreted by mutant producer cells. It may also contribute to biocompatibility, e.g. by preventing sequestration of biomolecules necessary for cellular growth, gene expression and/or signalling.

The surfactant may also function to isolate the individual microdroplets (and the corresponding microcultures), so that they serve as individual microvessels for co-culture of mutant producer and target cells. In such embodiments, the surfactant may be both hydrophobic and lipophobic, and so exhibit low solubility for the biological reagents of the aqueous phase while inhibiting molecular diffusion between microdroplets.

In some embodiments, the surfactant stabilizes (i.e. prevents coalescence) of a single emulsion comprised of droplets of aqueous media (containing encapsulated cells) dispersed in an oil phase for the duration of the incubation step. Similarly, the surfactant may stabilize droplets of aqueous media (containing encapsulated cells) in a double water-in-oil-in-water (W/O/W) type emulsion for the duration of the incubation step.

Thus, the surfactant may stabilize the microdroplet library under the conditions employed for co-culture of the single mutant producer cell and target cell(s), and so may stabilize the microdroplet at the selected incubation temperature (e.g. about 25° C.) for the selected incubation time (e.g. for at least an hour, and in some embodiments for up to 14 days).

Stabilization performance can be monitored by e.g. phase-contrast microscopy, light scattering, focused beam reflectance measurement, centrifugation and/or rheology.

Examples of suitable surfactants include: Abil WE 09 (Evonik—a 1:1:1 mixture of cetyl PEG/PPG 10/1 dimethicone, polyglyceryl-4 isostearate and hexyl laurate); Span® 80; Tween® 20; Tween® 80 and combinations thereof.

Oils for Use in Emulsion Co-Encapsulation

While it will be appreciated than any liquid immiscible with the aqueous growth medium may be used in the formation of microdroplet emulsions for use according to the invention, the immiscible fluid is typically an oil.

Preferably, an oil is selected having low solubility for biological components of the aqueous phase. Other preferred functional properties include good solubility for gases, the ability to inhibit molecular diffusion between microdroplets and/or combined hydrophobicity and lipophobicity. The oil may be a hydrocarbon oil, but preferred are light mineral oils, fluorocarbon or ester oils. Mixtures of two or more of the above-described oils are also preferred.

Examples of suitable oils include: diethylhexyl carbonate (Tegosoft DEC (Evonik)); light mineral oil (Fisher); and combinations thereof.

Processes for Microdroplet Emulsification

A wide range of different emulsification methods are known to those skilled in the art, any of which may be used to create the microdroplets of the invention.

Many emulsification techniques involve mixing two liquids in bulk processes, often using turbulence to enhance drop breakup. Such methods include vortexing, sonication, homogenization or combinations thereof.

In these “top-down” approaches to emulsification, little control over the formation of individual droplets is available, and a broad distribution of microdroplet sizes is typically produced. Alternative “bottom up” approaches operate at the level of individual drops, and may involve the use of microfluidic devices. For example, emulsions can be formed in a microfluidic device by colliding an oil stream and a water stream at a T-shaped junction: the resulting microdroplets vary in size depending on the flow rate in each stream.

A preferred process for producing microdroplets for use according to the invention comprises flow focusing (as described in e.g. Anna et al. (2003) Appl. Phys. Lett. 82(3): 364-366). Here, a continuous phase fluid (focusing or sheath fluid) flanking or surrounding the dispersed phase (focused or core fluid), produces droplet break-off in the vicinity of an orifice through which both fluids are extruded. A flow focusing device consists of a pressure chamber pressurized with a continuous focusing fluid supply. Inside, one or more focused fluids are injected through a capillary feed tube whose extremity opens up in front of a small orifice linking the pressure chamber with the external ambient environment. The focusing fluid stream moulds the fluid meniscus into a cusp giving rise to a steady micro or nano-jet exiting the chamber through the orifice; the jet size is much smaller than the exit orifice. Capillary instability breaks up the steady jet into homogeneous droplets or bubbles.

The feed tube may be composed of two or more concentric needles and different immiscible liquids or gases be injected leading to compound drops. Flow focusing ensures an extremely fast as well as controlled production of up to millions of droplets per second as the jet breaks up.

Other possible microfluidic droplet-forming techniques include pico-injection, whereby oil in water droplets are first formed and then pass down a T junction, along the top of the T and then inner aqueous phase is injected into the oil droplet creating a double emulsion.

In all cases, the performance of the selected microdroplet forming process may be monitored by phase-contrast microscopy, light scattering, focused beam reflectance measurement, centrifugation and/or rheology.

Fluorescence-Activated Droplet Sorting

As explained herein, the methods of invention are suitable for high-throughput screening, since they involve compartmentalizing the screening assay in tiny volumes of growth medium in the form of discrete microdroplets. This permits each microdroplet to be treated as a separate culture vessel, permitting rapid screening of large numbers of individual liquid co-cultures using established microfluidic and/or cell-sorting methodologies.

Thus, following the co-encapsulation step, the resultant microdroplets may be sorted by adapting well-established fluorescence-activated cell sorting (FACS) devices and protocols. This technique has been termed Fluorescence-Activated Droplet Sorting (FADS), and is described, for example, in Baret et al. (2009) Lab Chip 9: 1850-1858.

FADS may be used to manipulate the microdroplets at any stage after their formation in the methods of the invention, but are preferably used at least to screen the library of microcultures those in which target cells have been outgrown or overgrown to extinction by mutant producer cells. FADS may also be used during the co-encapsulation step, for example to eliminate empty microdroplets which do not contain mutant producer and/or target cell(s).

Either or both of the mutant producer cells and target cells may be fluorescently labelled to enable FADS. A variety of fluorescent proteins can be used as labels for this purpose, including for example the wild type green fluorescent protein (GFP) of Aequorea victoria (Chalfie et al. 1994, Science 263:802-805), and modified GFPs (Heim et al. 1995, Nature 373:663-4; PCT publication WO 96/23810). Alternatively, DNA2.0's IP-Free© synthetic non-Aequorea fluorescent proteins can be used as a source of different fluorescent protein coding sequences that can be amplified by PCR or easily excised using the flanking Bsal restriction sites and cloned into any other expression vector of choice.

Transcription and translation of this type of reporter gene leads to accumulation of the fluorescent protein in the cells, so rendering them amenable to FADS.

Incubation

The incubation conditions and the nature of the aqueous growth medium are selected according to the nature of the selected producer and target cells, and those skilled in the art will be able to readily determine appropriate media, growth temperatures and duration of incubation.

For example, mesophilic organisms will generally be incubated at 15° C.-42° C., while moderate thermophiles will be cultured at higher temperatures (typically 40° C.-60° C.).

Thermophiles and hyperthermophiles will be cultured at even higher temperatures (typically 60° C.-80° C. and 80° C.-98° C., respectively).

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1: Encapsulation Using Single Emulsion

Oil Mix for Continuous Phase
73% Tegosoft DEC (Evonik), 20% light mineral oil (Fisher), 7% Abil WE09 (Evonik) (surfactant)
70% Tegosoft DEC (Evonik), 20.3% light mineral oil (Fisher) 4.5% Span-80 (surfactant), 4.8% Tween20 (surfactant)
90% light mineral oil, 10% Span-80 (surfactant)

All oil mixes need to be made at least 30 minutes prior to use but can be kept indefinitely.

Aqueous Growth Media for Dispersed Phase

This may be selected from the following:
SOC broth (20 g/L tryptone, 5 g/L yeast extract 10 mM NaCl, 2.5 mM KCl, 10 mM MgCl₂, 10 mM MgSO₄ and 20 mM glucose)
SOC+5% Glycerol
LB broth (10 g/L Peptone, 5 g/L Yeast extract, 10 g/L NaCl)
2×YT (16 g/L tryptone, 10 g/L Yeast Extract, 5 g/NaCl)
2×YT+0.5% Glucose and 5% Gylcerol
2×YT+5% glycerol
Peptone glycerol media 5 g/L peptone, 5% glycerol

The inclusion of glycerol as a carbon source can facilitate microdroplet formation by vortexing.
1. 0.5 ml to 10 ml of growth media containing the producer and target cells at appropriate cell numbers to give <1 producer and then ≧1 target cell per droplet produced. This is aliquoted into a suitable vessel (Eppendorf tube 1.5 or 2 ml 15 ml or 50 ml falcon or 24 well plate are acceptable).
2. This growth media is then overlaid with 1-2× volume of the oil mix.
3. This is the vortexed for between 3-6 minutes (usually 5 minutes) at 12,000-18,000 rpm. The speed varies by oil and media used as well as the desired droplet size as a general rule the higher the speed and the longer the vortexing the smaller the average droplet size (although a range of sizes are always generated by this method).
4. The integrity of the droplets and presence of cells within is screened visually under a microscope using phase contrast. Alternative visualisation methods can be used e.g. fluorescence.
5. Droplets are then incubated at appropriate temperature (usually 37° C., but they are stable between 4° C. and 95° C.) for time periods of 1 h to days.
6. Droplets can then be sorted by FADS if required and then desired cells recovered from the droplets.
7. To break open droplets additional surfactant is added (e.g. 1% SDS for Tegosoft oil, but any appropriate surfactant such as Tweens, Sarcosyl etc. can be used) this disrupts droplet integrity releasing cells.
8. Some oil mixes can be lysed by freezing the solution disrupting the droplets.
9. Cells can then be recovered by centrifugation, or the sample can be applied directly to columns for DNA extractions.

### Example 2: Encapsulation Using Double Emulsion

Aqueous Growth Media

As in Example 1 (above).

Mixes Used to Produce Outer Aqueous Phase
Phosphate buffered Saline (PBS; 10 mM phosphate buffer, 137 mM NaCl) with 2% Tween-20
PBS with 2% Tween-80 (surfactant)

Oil Mix for Droplet Shells

As for the oil mixes set out in Example 1 (above).
1. 0.5 ml to 10 ml of growth media containing the producer and target cells at appropriate cell numbers to give <1 producer and then ≧1 target cell per droplet produced. This is aliquoted into a suitable vessel (Eppendorf tube 1.5 or 2 ml 15 ml or 50 ml falcon or 24 well plate are acceptable).
2. This growth media is then overlaid with 1-2× volume of the oil mix.
3. This is the vortexed for between 3-6 minutes (usually 5 minutes) at 12,000-18,000 rpm. The speed varies by oil and media used as well as the desired droplet size as a general rule the higher the speed and the longer the vortexing the smaller the average droplet size although a range of sizes are always generated by this method.
4. The integrity of the droplets and presence of cells within is screened visually under a microscope using phase contrast. Alternative visualisation methods can be used e.g. fluorescence.
5. A second vortexing step is then introduced to generate the double emulsion. A volume equal to the oil volume of second aqueous phase with surfactant is added to the single emulsion. This is then vortexed as but at reduce rpm (6,000 to 10,000 rpm) for just 2-3 minutes. The formation of double emulsion droplets can then be visualised on a microscope as before.
6. Droplets are then incubated at appropriate temperature (usually 37° C., but they are stable between 4° C. and 95° C.) for time periods of 1 h to days.
7. Droplets can then be sorted by FADS if required and then desired cells recovered from the droplets.
8. To break open droplets additional surfactant is added (e.g. 1% SDS for Tegosoft oil, but any appropriate surfactant such as Tweens, Sarcosyl or similar can be used) this disrupts droplet integrity releasing cells.
9. Some oil mixes can be lysed by freezing the solution disrupting the droplets.
10. Cells can then be recovered by centrifugation, or the sample can be applied directly to columns for DNA extractions.

### Example 3: Production of Microdroplets Using a Microfluidic Chip

Droplet generation in a microfluidic allows creation of single and double emulsions. In its simplest form double emulsions are formed by sequential formation of and oil in water droplet and then formation of a second aqueous layer by the same methodology.

Alternative methods involve simultaneous co-encapsulation of aqueous phase in oil and then the oil-aqueous droplet in a secondary continuous phase of aqueous media. Droplet formation on a chip leads to generation of highly uniform sized droplets. Size is directly related to the size of the channels on the fluidic and the flow rates used to generate droplets. Microfluidic droplet formation allows the use of novel oil and surfactant mixes not available when producing droplets in bulk “top-down” approaches.

The producer and target cells in suitable growth media are mixed at appropriate ratios to allow for producer per droplet and then ≧1 target cell per droplet. This aqueous mixture of cells is then pumped through a microfluidic device with geometry that allows the formation of water in oil single emulsion droplets, or double emulsion droplets, as detailed below.

These droplets are collected in a vessel suitable for incubation and the incubated at appropriate temperature (usually 37° C., but they are stable between 4° C. and 95° C.) for time periods of 1 h to ≧14. days.

Droplets can then be sorted by FADS if required and then desired cells recovered from the droplets.

To break open droplets, additional surfactant is added (e.g. 1% SDS for Tegosoft oil, but any appropriate surfactant such as Tweens, Sarcosyl etc. can be used) to disrupt droplet integrity, so releasing the cells. Some oil mixes can be lysed by freezing the solution disrupting the droplets.

Cells can then be recovered by centrifugation, or the sample can be applied directly to columns for DNA extractions.

### Example 4: Production of Double Emulsion Microdroplets Using Flow Focusing

Here, oil flows from the sides into a hydrophobic channel causing pinch off of the aqueous phase generating the water in oil droplets. This method allows good size control and 1000's of droplets per second to be generated depending on flow rates of the second liquid phases.

It is possible to use two such chips connected in series to generate double emulsions: the first droplets are aqueous phase in oil which are pushed through a second chip as if aqueous phase, the side flowing oil being replaced with the secondary aqueous phase.

It is also possible to integrate both steps into a single chip and junction interface; whereby water in oil droplets are formed and immediately surrounded in the outer aqueous phase to generate the W/O/W double emulsion.

The hydrophilic and hydrophobic coatings are inverted on the second chip, thus the second chip has the same geometry, but the opposite surface coatings to facilitate flow of aqueous phase as the carrier/sheath fluid. This permits a two stage encapsulation: the first to generate single phase emulsions which are used for incubation and co-culturing of the producer and target cells, and a second stage in which the microcultures are converted into double emulsions for subsequent FADS.

### Example 5: Co-Culture Conditions for Prokaryotic and Eukaryotic Cells

Balanced co-culture of bacterial and eukaryotic cells (i.e. culture conditions under which the doubling rates of both prokaryotic and eukaryotic cells are not so different as to result in rapid overgrowth of one class of cells) can be readily achieved by selection of appropriate culture media and incubation conditions (including inter alia the extent of aeration and temperature of incubation).

Table 1 below shows the growth of soil bacteria used for co-culture in commercially available tissue culture media and under conditions identical to those used to culture mammalian cells in commonly used mammalian culture media (i.e. at 37° C.). Table 2 shows the published growth rates of a selection of eukaryotic cell-lines under these conditions. Table 3 shows the growth of soil bacteria used for co-culture in commercially available tissue culture media at 30° C. (rather than 37° C.), but otherwise under conditions identical to those used to obtain the data shown in Table 1.

TABLE 1
Growth at 37° C.

| Strain | Pseudomonas | Pseudomonas | Pseudomonas | Streptomyces | Streptomyces |
|---|---|---|---|---|---|
| Media | flourescens | putida | mandelii | venezuelae | 14100 |
| Freestyle | 43.6 hours | 45.8 hours | 51.1 hours | 33.07 hours | 22.7 hours |
| DMEM + | 34.9 hours | 48.7 hours | 34.8 hours | 27.0 hours | 29.1 hours |
| 10% FBS | | | | | |
| RPMI + | 33.0 hours | 32.75 hours | 35.2 hours | 38.8 hours | 29.1 hours |
| 10% FBS | | | | | |

TABLE 2
Representative growth rates of eukaryotic cells

| | | | | | Human | Murine 264 | |
|---|---|---|---|---|---|---|---|
| Cell lines | HEK-293 | COS-7 | Jurkat | Hybridoma | A549 | cell line | B16F10 |
| Doubling | 10 | 18 | 48 | 14 | 27 | 11 | 12 |
| time (h) | | | | | | | |

TABLE 3
Growth at 30° C.

| Strain | Pseudomonas | Pseudomonas | Pseudomonas | Streptomyces | Streptomyces |
|---|---|---|---|---|---|
| Media | flourescens | putida | mandelii | venezuelae | 14100 |
| Freestyle | 15.7 hours | 21.0 hours | 18.5 hours | 22.4 hours | 13.3 hours |
| DMEM | 19.5 hours | 19.3 hours | 23.0 hours | 18.7 hours | 16.1 hours |
| RPMI | 23.9 hours | 22.5 hours | 21.1 hours | 52.0 hours | 20.4 hours |

Growth rate was calculated by growing fresh overnight cultures of each culture at 30° C. in the desired mammalian culture media. This was then diluted 1 in 50 into fresh tissue culture media and the OD₆₀₀ then measured at various time points during growth at 37° C.+5% CO₂ or at 30° C. The growth rate was calculated from the doubling time during exponential growth of the culture, and based on individual cultures grown in triplicate.

As can be seen from the above data, the relative growth rates of prokaryotic and eukaryotic cells in co-culture can be readily controlled to achieve balanced co-cultures by inter alia the selection of appropriate culture conditions, including incubation temperature.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method for screening mutant prokaryotic cells to identify producers of a cytotoxic agent active against a target cell, the method comprising the steps of:
(a) providing cells of a producer prokaryotic species;
(b) generating a pool of mutant producer cells by transposon mutagenesis of the cells of step (a) with an activating transposon (Tn_{A}), wherein the Tn_{A} comprises an outward-facing promoter (Tn_{A}P) capable of increasing transcription of a gene at or near its insertion site in the DNA of said producer cells;
(c) co-encapsulating individual members of the pool of step (b) with one or more target cells in microdropiets, the microdroplets comprising a volume of aqueous growth media suspended in an immiscible carrier liquid, thereby generating a library of microdropiets each comprising a single mutant producer cell and one or more target cell(s);
(d) incubating the microdroplet library of step (c) under conditions suitable for co-culture of the single mutant producer cell and target cell(s) to produce a library of microcultures, whereby mutant producer cells producing a cytotoxic agent active against the target cell(s) outgrow target cells in each microculture; and
(e) screening the library of microcultures of step (d) for microcultures in which target cells have been outgrown or overgrown to extinction by mutant producer cells.

2. The method of claim 1 further comprising the step of sequencing the DNA of mutant producer cells in microdroplets in which target cells have been outgrown or overgrown to extinction by mutant producer cells.

3. The method of claim 2 wherein DNA adjacent or near the insertion site of the Tn_{A} is sequenced.

4. The method of claim 3 wherein the sequencing of DNA adjacent or near the insertion site of the Tn_{A} comprises selective amplification of transposon-cellular DNA junctions.

5. The method of claim 3 wherein the sequencing comprises high-throughput massively parallel sequencing, for example selected from: (a) sequencing-by-synthesis (SBS) biochemistry; and/or (b) nanopore sequencing; and/or (c) tunnelling current sequencing; and/or (d) pyrosequencing; and/or (e) sequencing-by-ligation (SOLiD sequencing); and/or (f) ion semiconductor; and/or (g) mass spectrometry sequencing

6. The method of claim 2 wherein about 25, 50, 75, 100 or greater than 100 base pairs of DNA adjacent or near the Tn_{A} insertion site are sequenced.

7. The method of claim 2 wherein the sequenced DNA is 5′ and/or 3′ to the Tn_{A} insertion site.

8. The method of claim 2 further comprising the step of sequencing mRNA transcripts produced by Tn_{A}P in mutant producer cells in microdroplets in which target cells have been outgrown or overgrown to extinction by mutant producer cells to produce an mRNA transcript profile.

9. The method of claim 8 wherein said mRNA transcript profile comprises a determination of:
(a) the sequences of said mRNA transcripts produced by Tn_{A}P; and/or
(b) the start and finish of mRNA transcripts produced by Tn_{A}P; and/or
(c) the lengths of said mRNA transcripts produced by Tn_{A}P; and/or
(d) the relative abundance of said mRNA transcripts produced by Tn_{A}P; and/or
(e) the site of transcription on the cellular DNA; and/or
(f) whether the mRNA transcripts produced by Tn_{A}P is sense or antisense with respect to the cellular DNA; and/or
(g) whether the mRNA transcripts produced by Tn_{A}P correspond to ORFs with respect to the cellular DNA; and/or
(h) whether the mRNA transcripts produced by Tn_{A}P encode prokaryotic proteins and/or protein domains.

10. The method of claim 1 wherein the microdroplets are substantially spherical and have a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm.

11. The method of claim 1 wherein the microdroplets comprise a volume of aqueous growth media in the gel state.

12. The method of claim 1 wherein the microdroplets comprise a volume of aqueous growth media in the liquid state.

13. The method of claim 1 wherein the microdroplets comprise an inner core of aqueous growth media enveloped in an outer oil shell, the carrier liquid being a continuous aqueous phase.

14. The method of claim 13 wherein the inner aqueous core has a diameter of: (a) 10 μm to 500 μm; (b) 10 μm to 200 μm; (c) 10 μm to 150 μm; (d) 10 μm to 100 μm; (e) 10 μm to 50 μm; or (f) about 100 μm.

15. The method of claim 13 wherein the outer oil shell has a thickness of: (a) 10 μm to 200 μm; (b) 10 μm to 200 μm; (c) 10 μm to 150 μm; (d) 10 μm to 100 μm; (e) 10 μm to 50 μm; or (f) about 100 μm.

16. The method of claim 1 wherein the carrier liquid is a water-immiscible liquid.

17. The method of claim 16 wherein the water-immiscible liquid is an oil, for example selected from: (a) a hydrocarbon oil; (b) a fluorocarbon oil; (c) an ester oil; (d) an oil having low solubility for biological components of the aqueous phase; (e) an oil which inhibits molecular diffusion between microdroplets; (f) an oil which is hydrophobic and lipophobic; (g) an oil having good solubility for gases; and/or (h) combinations of any two or more of the foregoing.

18. The method of claim 16 wherein the microdroplets are comprised in a W/O emulsion wherein the microdroplets constitute an aqueous, dispersed, phase and the carrier liquid constitutes a continuous oil phase.

19-66. (canceled)

67. A method of identifying a cytotoxic agent comprising screening mutant bacteria to identify producers of a cytotoxic agent active against a target cell according to a method as defined in claim 1.

68. A process for producing a cytotoxic agent comprising the method as defined in claim 1.

69-70. (canceled)
